(19) United States
(12) Patent Application Publication
(10) Pub. No.: US 2026/0115211 A1
(43) Pub. Date: Apr. 30, 2026

(54) METHOD FOR PREP ARING VEGETABLE OIL EMULSION USING CANNABIDIOL OIL

(71) Applicant: ADHOME CO.,LTD., Yongin-si Gyeonggi-do (KR)

(72) Inventor: Jae Hwa TARK, Seoul (KR)

(21) Appl. No.: 18/281,325

(22) PCT Filed: Dec. 16, 2020
(86) PCT No.: PCT/KR2020/018408

§ 371 Date: Feb. 20, 2025

(51) Int. Cl. A61K 31/00; A61K 9/107; A61K 41/00; A61K 47/44

(52) U.S. Cl. CPC A61K 31/658; A61K 9/107; A61K 41/00; A61K 47/44

(57) ABSTRACT

A method of preparing a vegetable oil emulsion using a cannabidiol oil includes adding ethanol to a gel-type cannabidiol oil while heating the cannabidiol oil in a water bath (S100), continuously performing the heating in the water bath to reduce a particle size of the cannabidiol oil in the result (S200), stopping the heating in the water bath and adding purified water to the result (S300), and heating the result again in a water bath while slowly elevating a temperature to completely remove the ethanol (S400).

### BACKGROUND

The present invention relates to a method of preparing a vegetable oil emulsion using a cannabidiol oil, and more specifically, to a method of preparing a vegetable oil emulsion using a cannabidiol oil that has excellent oil emulsion conversion rate and greatly prevents layer separation between the oil and purified water without an emulsifier or surfactant.

A cannabidiol (CBD) oil is extracted from a plant called “cannabis plant” or “hemp” by ethanol extraction, olive extraction, or carbon dioxide (CO₂) extraction and maintains a gel state at room temperature.

Meanwhile, such cannabidiol (CBD) oil is reported to be highly effective in inhibiting cancer cells, relieving pain, treating depression, ameliorating insomnia, stimulating appetite in cancer patients, relieving symptoms of epilepsy, and ameliorating eczema and atopy, and to contain various vitamins such as A, B, D, and E, and to be rich in other nutrients.

Conventionally, a cannabidiol (CBD) oil has been used in combination with hemp seed oil or coconut oil. When cannabidiol (CBD) oil is used alone, layer separation occurs, thus being inapplicable to commercialization of various products containing the same.

In other words, when a cannabidiol (CBD) oil is mixed with a polar solvent such as water, homogeneous mixing therebetween is very difficult due to the characteristics thereof. Even if a cannabidiol (CBD) oil is mixed with a polar solvent such as water, disadvantageously, layer separation occurs between water and the oil within a short time.

Meanwhile, Patent Document 1 below discloses a cosmetic composition for ameliorating atopy containing cannabidiol or a derivative thereof, but the cannabidiol contained in the cosmetic composition of Patent Document 1 is disadvantageously easily separated from other components of the cosmetic composition.

Therefore, there is an urgent need to develop a novel method of emulsifying a cannabidiol oil that has excellent oil-emulsion conversion efficiency and prevents layer separation between cannabidiol and a polar solvent such as water even after a long period of time.
Patent Document 1: Korean Patent Laid-open Publication No. 10-2019-0098648 (Aug. 22, 2019)

### SUMMARY OF THE INVENTION

Therefore, the present invention has been made in view of the above problems, and it is an object of the present invention to provide a method of preparing a vegetable oil emulsion using a cannabidiol oil that has excellent oil-emulsion conversion efficiency and prevents layer separation between cannabidiol and a polar solvent such as water even after a long period of time.

In accordance with the present invention, the above and other objects can be accomplished by the provision of a method of preparing a vegetable oil emulsion using a cannabidiol oil including adding ethanol to a gel-type cannabidiol oil while heating the cannabidiol oil in a water bath (S100), continuously performing the heating in the water bath to reduce a particle size of the cannabidiol oil in the result (S200), stopping the heating in the water bath and adding purified water to the result (S300), and heating the result again in a water bath while slowly elevating a temperature to completely remove the ethanol (S400).

Step (S100) may be performed by heating the result in the water bath at a temperature of 50 to 70° C.

Step (S200) may be performed until an average particle size of the cannabidiol oil reaches 20 nm or less.

A temperature of the purified water added in step (S300) may be 15 to 20° C.

The method may further include applying ultrasonic waves to the cannabidiol oil mixed with purified water (S500) after step (S400).

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram illustrating a process of preparing a vegetable oil emulsion using a cannabidiol oil according to an embodiment of the present invention.

### DETAILED DESCRIPTION OF THE INVENTION

Prior to the detailed description of the present invention, it should be noted that the terms or words used in this disclosure and claims are not limited to ordinary or dictionary meanings and are interpreted as having meanings and concepts consistent with the technical spirit of the present invention based on the principle that the concept of terms can be adequately defined to describe the invention in the best possible way. Therefore, the configuration of the embodiments described herein is provided as only a preferred example of the present invention and does not represent all of the technical spirit of the present invention. Accordingly, those skilled in the art will appreciate that the prevent invention includes various equivalents and modifications that can replace the embodiments at the filing time of the present application.

The present invention relates to a method for preparing a vegetable oil emulsion using a cannabidiol oil, and more specifically, a method of preparing a vegetable oil emulsion using a cannabidiol oil that has excellent oil emulsion conversion rate and greatly prevents layer separation between an oil and purified water without an emulsifier or surfactant.

As used herein, the term “emulsion” may be interpreted as meaning a solution in which two or more substances that are difficult to mix with each other due to physical or chemical properties are mixed to homogeneity. For example, an oil emulsion means a solution in which water and oil are homogeneously mixed with each other.

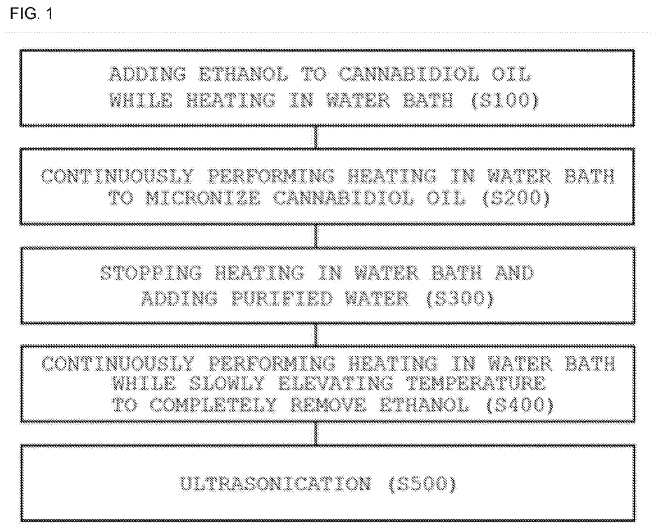

FIG. 1 is a schematic diagram illustrating a process of preparing a vegetable oil emulsion using a cannabidiol oil according to an embodiment of the present invention.

Referring to FIG. 1, the method of preparing the vegetable oil emulsion using the cannabidiol oil according to an embodiment of the present invention includes adding ethanol to a gel-type cannabidiol oil while heating the cannabidiol oil in a water bath (S100), continuously performing the heating in the water bath to reduce a particle size of the cannabidiol oil in the result (S200), stopping the heating in the water bath and adding purified water to the result (S300), and heating the result again in a water bath while slowly elevating a temperature to completely remove the ethanol (S400).

A cannabidiol (CBD) oil is extracted from a plant called “cannabis plant” or “hemp” by ethanol extraction, olive extraction, or carbon dioxide (CO₂) extraction and maintains a gel state at room temperature of about 15 to about 25° C.

In the prior art, a cannabidiol (CBD) oil that maintains the gel state thereof at room temperature has been used in combination with hemp seed oil or coconut oil in order for the cannabidiol (CBD) oil to be applied to various products.

However, the mixture of the cannabidiol oil with other vegetable oils causes problems of inevitably reducing the oil-emulsion conversion efficiency and the purity of the cannabidiol oil in the emulsion.

Accordingly, the present inventors developed a novel method of emulsifying a cannabidiol oil with high emulsification efficiency that is capable of emulsifying the cannabidiol oil at a high emulsification efficiency, and of preventing separation between oil and water layers without mixing the cannabidiol oil with other vegetable oils.

The process described below is performed in order to reduce the viscosity of cannabidiol oil, which maintains a gel state at room temperature and thereby convert the cannabidiol oil to a liquid.

First, ethanol is added to a gel-type cannabidiol oil while heating the cannabidiol oil in a water bath (S100).

In step S100, a heating container containing a gel-phase cannabidiol oil is injected into a water bath heater and the water bath heater is operated to convert the cannabidiol oil from the gel into a liquid.

This water bath heating process is performed while maintaining the water temperature of the water bath heater at 50 to 70° C.

Meanwhile, in the process of performing step S100, ethanol is added to the gel-phase cannabidiol oil heated in the water bath.

As such, when ethanol is added in step S100, the gel-phase cannabidiol oil undergoes an emulsification process, and thus the emulsified cannabidiol oil is not readily reduced to the gel phase.

That is, for example, when 0.5 to 100 kg of ethanol is added to 10 kg of cannabidiol oil, the time for which the cannabidiol oil is dissolved in the ethanol solvent takes about 30 minutes to 2 hours depending on the amount of the solvent. However, when ethanol is added while heating the oil in the water bath, the dissolution time can be reduced, and at the same time, layer separation after solubilization can be greatly reduced.

The amount of ethanol added to 10 kg of the cannabidiol oil in step (S100) is 0.5 to 100 kg and the ethanol is added is preferably two or more times in a divided portion. After each portion of ethanol is added, it is preferable to homogeneously stir the cannabidiol oil containing ethanol with a glass rod or the like.

When the water temperature of the water bath is set to 60° C., the amount of cannabidiol present in the container is 1 kg, the total amount of ethanol added 5 times at a regular interval of 200 g is 1 kg, and step (S100) is performed for about 10 to about 30 minutes (Condition 1).

Then, the heating in the water bath and stirring are continuously performed to reduce a particle size of the cannabidiol oil to which the ethanol is added (S200).

During step S200, ultrasonic waves are applied to the container containing cannabidiol and ethanol and at the same time, heating of the water bath is continuously performed.

During step S200, the mixture of the cannabidiol oil, ethanol and purified water is continuously and homogeneously mixed while slowly adding purified water having a temperature of 15 to 20° C. thereto.

When a solution containing a cannabidiol oil and ethanol is added to purified water and ultrasonic waves are applied to an aqueous solution containing 0.1 to 3% by weight of cannabidiol oil, the ethanol evaporates during ultrasonic application to obtain a pure aqueous cannabidiol solution.

In this way, in the case of Condition 1, the ultrasonic waves applied in step S200 have a frequency of 80 to 120 kHz, the ultrasonic application time is 20 to 120 minutes, and the temperature of the mixture of cannabidiol and ethanol is maintained at 40 to 60° C. (first ultrasound application).

In this way, after step S200 performed along with the application of ultrasonic waves, the average diameter of the cannabidiol oil particles is reduced to about 20 nm or less.

Then, when the average diameter of the cannabidiol oil particles decreases to about 20 nm or less, the heating of the water bath is stopped and purified water is added thereto (S300).

Meanwhile, the temperature of the purified water added in step S300 is preferably 15 to 20° C.

That is, the temperature of the mixture of cannabidiol and ethanol is maintained at 40 to 60° C. while ultrasonic waves are applied in step S200. Therefore, the temperature of the mixture of cannabidiol and ethanol immediately after the application of the ultrasonic wave is finished is 40 to 60° C.

At this time, the mixture of cannabidiol, ethanol and purified water is continuously and homogeneously mixed while slowly adding purified water having a temperature of 15 to 20° C. thereto.

The total amount of purified water that is added is preferably about 30 to 1,000 kg in the condition 1 (cannabidiol 1 kg, ethanol 1 kg).

Meanwhile, the resulting product is again heated while performing ultrasonication to completely remove the ethanol (S400).

More specifically, step (S400) is performed while gradually elevating the temperature at a rate of 5° C./min based on the condition 1 until the bath temperature is 75 to 80° C. and performing continuously ultrasonication for about 2 to 3 hours based on condition 1 above, to induce complete evaporation of the ethanol from the mixture.

Meanwhile, preferably, ultrasonic waves are preferably applied to the cannabidiol oil mixed with purified water in the state where the added ethanol is completely removed (S500).

In this way, in the case of Condition 1, the ultrasonic waves applied in step S500 have a frequency of 150 to 180 kHz, the ultrasonic application time is 10 to 20 minutes, and the temperature of the mixture of cannabidiol and ethanol is maintained at 70 to 75° C. (second ultrasound application).

Hereinafter, specific examples according to the present invention and various comparative examples to compare the effects therebetween will be described.

### EXAMPLE

A container containing 1 kg of a gel-phase cannabidiol oil was injected into a water bath heater containing water for a water bath and the water temperature of the water bath heater was set to 60° C. In this way, the cannabidiol oil was homogeneously stirred for 1 hour in a state where the water temperature of the water bath heater was maintained at 60° C. Then, the water temperature of the water bath was maintained at 60° C., 1 kg of ethanol was added 5 times in a divided portion of 200 g to a container containing the diluted liquid cannabidiol oil, and the cannabidiol oil and ethanol were homogeneously stirred for 1 hour whenever 200 g of ethanol was added. Then, first ultrasonication of applying ultrasonic waves to a container containing cannabidiol and ethanol to micronize cannabidiol particles was performed. The first ultrasonication was performed under various conditions while varying the frequency of the ultrasonic wave from 80 to 120 kHz, setting the duration of ultrasonication to 30 minutes, and changing the temperature of the mixture of cannabidiol and ethanol during the ultrasonication from 40 to 60° C. Then, when the average diameter of cannabidiol oil particles decreased to about 20 nm or less through the first ultrasonication, the heating of the water bath was stopped and 10 kg of purified water was added thereto. At this time, the temperature of the purified water added was set to 15° C. and the mixture of cannabidiol, ethanol, and purified water was continuously and homogeneously mixed while the purified water was gradually added thereto. Meanwhile, after the purified water was added in this way, when the temperature of the entire mixture was cooled to about 20° C., the result was heated in the water bath again while slowly elevating the temperature to completely remove the added ethanol. The ethanol removal was performed while gradually increasing the temperature at a rate of 5° C./min until the water bath temperature reached 78° C. When the water bath temperature reached 78° C., the water bath heating was continued for about 2 hours. Then, second ultrasonication of applying ultrasonic waves again to the cannabidiol oil mixed with purified water was performed in a state in which the ethanol added was completely removed through the process. Specifically, the frequency of the ultrasonic wave applied in the second ultrasonication process was set to 170 kHz, the ultrasonication time was 15 minutes, and the temperature of the mixture of cannabidiol and ethanol was maintained at 70° C.

### Comparative Example

A container containing 1 kg of a gel-phase cannabidiol oil was injected into a water bath heater containing water for a water bath and the water temperature of the water bath heater was set to 60° C. In this way, the cannabidiol oil was homogeneously stirred for 1 hour in a state where the water temperature of the water bath heater was maintained at 60° C. Then, the water temperature of the water bath was maintained at 60° C., 1 kg of ethanol was added 5 times in a divided portion of 200 g to a container containing the diluted liquid cannabidiol oil, and the cannabidiol oil and ethanol were homogeneously stirred for 1 hour whenever 200 g of ethanol was added. Then, first ultrasonication of applying ultrasonic waves to a container containing cannabidiol and ethanol to micronize cannabidiol particles was performed. The first ultrasonication was performed under various conditions while varying the frequency of the ultrasonic wave from 20 to 79 kHz and from 121 to 150 kHz, setting the duration of ultrasonication to 30 minutes, and changing the temperature of the mixture of cannabidiol and ethanol during the ultrasonication from 10 to 39° C. and 61 to 80° C. Then, when the average diameter of cannabidiol oil particles decreased to about 20 nm or less through the first ultrasonication, heating of the water bath was stopped and 10 kg of purified water was added thereto. At this time, the temperature of the purified water was set to 15° C. and the mixture of cannabidiol, ethanol, and purified water was continuously and homogeneously mixed while the purified water was gradually added thereto. Meanwhile, after the purified water was added in this way, when the temperature of the entire mixture was cooled to about 20° C., the result was heated in the water bath again while slowly elevating the temperature to completely remove the added ethanol. The ethanol removal was performed while gradually increasing the temperature at a rate of 5° C./min until the water bath temperature reached 78° C. When the water bath temperature reached 78° C., the water bath heating was continued for about 2 hours. Then, second ultrasonication of applying ultrasonic waves again to the cannabidiol oil mixed with purified water was performed in a state in which the ethanol added was completely removed through the process. Specifically, the frequency of the ultrasonic wave applied in the second ultrasonication process was set to 170 kHz, the ultrasonication time was 15 minutes, and the temperature of the mixture of cannabidiol and ethanol was maintained at 70° C.

### Experiment 1: Test for Measurement of Emulsification Efficiency

The ratio (%) at which the used cannabidiol oil is converted into an emulsion was measured using each of the oil emulsions prepared through Example and Comparative Example, and the results are shown in Table 1 below (the frequency of ultrasonic waves applied in the first ultrasonication and the temperature conditions of the mixture are changed).

TABLE 1

| Item | | 10° C. | 20° C. | 30° C. | 39° C. | 40° C. | 50° C. | 60° C. | 61° C. | 70° C. | 80° C. |
|---|---|---|---|---|---|---|---|---|---|---|---|
| 30 | kHz | 74 | 75 | 76 | 78 | 82 | 83 | 85 | 82 | 84 | 83 |
| 40 | kHz | 74 | 75 | 76 | 79 | 82 | 84 | 85 | 82 | 84 | 83 |
| 50 | kHz | 74 | 75 | 76 | 79 | 83 | 84 | 85 | 82 | 84 | 83 |
| 60 | kHz | 74 | 75 | 76 | 79 | 84 | 84 | 85 | 82 | 84 | 83 |
| 70 | kHz | 74 | 75 | 76 | 79 | 85 | 85 | 85 | 82 | 84 | 83 |
| 79 | kHz | 75 | 75 | 76 | 81 | 85 | 85 | 85 | 82 | 84 | 83 |
| 80 | kHz | 75 | 76 | 77 | 85 | 100 | 100 | 100 | 82 | 84 | 83 |
| 90 | kHz | 75 | 76 | 77 | 84 | 100 | 100 | 100 | 83 | 84 | 83 |
| 100 | kHz | 75 | 76 | 77 | 83 | 100 | 100 | 100 | 83 | 85 | 83 |
| 110 | kHz | 75 | 76 | 77 | 81 | 100 | 100 | 100 | 83 | 85 | 83 |
| 120 | kHz | 75 | 76 | 77 | 80 | 100 | 100 | 100 | 83 | 85 | 83 |
| 121 | kHz | 76 | 76 | 78 | 79 | 86 | 87 | 86 | 83 | 85 | 83 |
| 130 | kHz | 76 | 77 | 78 | 79 | 85 | 85 | 85 | 83 | 85 | 83 |
| 140 | kHz | 76 | 77 | 78 | 79 | 85 | 84 | 85 | 83 | 84 | 84 |
| 150 | kHz | 76 | 77 | 78 | 78 | 84 | 84 | 85 | 83 | 84 | 84 |

(unit: %)

As can be seen from the results of Table 1, when the frequency of the ultrasonic wave applied according to the embodiment of the present invention in the first ultrasonication was set to 80 to 120 kHz, and the temperature of the mixture of the cannabidiol and ethanol was set to 40 to 60° C., the emulsion conversion rate of cannabidiol oil was superb, i.e., 100%.

### Experiment 2: Test for Occurrence of Layer Separation at High Temperature

The oil emulsions prepared in Example and Comparative Example were allowed to stand at a temperature of 45° C. for 24 hours and a test was performed to determine whether or not layer separation between oil and purified water occurred, and the results are shown in Table 2 below (the frequency of ultrasonic waves applied in the first ultrasonication and the temperature conditions of the mixture are changed).

TABLE 2

| Item | | 10° C. | 20° C. | 30° C. | 39° C. | 40° C. | 50° C. | 60° C. | 61° C. | 70° C. | 80° C. |
|---|---|---|---|---|---|---|---|---|---|---|---|
| 30 | kHz | ◯ | ◯ | ◯ | ◯ | ◯ | ◯ | ◯ | ◯ | ◯ | ◯ |
| 40 | kHz | ◯ | ◯ | ◯ | ◯ | ◯ | ◯ | ◯ | ◯ | ◯ | ◯ |
| 50 | kHz | ◯ | ◯ | ◯ | ◯ | ◯ | ◯ | ◯ | ◯ | ◯ | ◯ |
| 60 | kHz | ◯ | ◯ | ◯ | ◯ | ◯ | ◯ | ◯ | ◯ | ◯ | ◯ |
| 70 | kHz | ◯ | ◯ | ◯ | ◯ | ◯ | ◯ | ◯ | ◯ | ◯ | ◯ |
| 79 | kHz | ◯ | ◯ | ◯ | ◯ | ◯ | ◯ | ◯ | ◯ | ◯ | ◯ |
| 80 | kHz | ◯ | ◯ | ◯ | ◯ | X | X | X | ◯ | ◯ | ◯ |
| 90 | kHz | ◯ | ◯ | ◯ | ◯ | X | X | X | ◯ | ◯ | ◯ |
| 100 | kHz | ◯ | ◯ | ◯ | ◯ | X | X | X | ◯ | ◯ | ◯ |
| 110 | kHz | ◯ | ◯ | ◯ | ◯ | X | X | X | ◯ | ◯ | ◯ |
| 120 | kHz | ◯ | ◯ | ◯ | ◯ | X | X | X | ◯ | ◯ | ◯ |
| 121 | kHz | ◯ | ◯ | ◯ | ◯ | ◯ | ◯ | ◯ | ◯ | ◯ | ◯ |
| 130 | kHz | ◯ | ◯ | ◯ | ◯ | ◯ | ◯ | ◯ | ◯ | ◯ | ◯ |
| 140 | kHz | ◯ | ◯ | ◯ | ◯ | ◯ | ◯ | ◯ | ◯ | ◯ | ◯ |
| 150 | kHz | ◯ | ◯ | ◯ | ◯ | ◯ | ◯ | ◯ | ◯ | ◯ | ◯ |

(◯: layer separation occurs, X: layer separation does not occur)

As can be seen from the results in Table 2, when the frequency of the ultrasonic wave applied according to the embodiment of the present invention in the first ultrasonication was set to 80 to 120 kHz, and the temperature of the mixture of the cannabidiol and ethanol was set to 40 to 60° C., the emulsified cannabidiol oil was not separated from the purified water at all.

### Experiment 3: Test for Occurrence of Layer Separation after Allowing to Stand at Room Temperature for Long Period of Time

The oil emulsions prepared in Example and Comparative Example were allowed to stand at a temperature of 30° C. for 3 months and a test was performed to determine whether or not layer separation between oil and purified water occurred, and the results are shown in Table 3 below (the frequency of ultrasonic waves applied in the first ultrasonication and the temperature conditions of the mixture are changed).

TABLE 3

| Item | | 10° C. | 20° C. | 30° C. | 39° C. | 40° C. | 50° C. | 60° C. | 61° C. | 70° C. | 80° C. |
|---|---|---|---|---|---|---|---|---|---|---|---|
| 30 | kHz | ◯ | ◯ | ◯ | ◯ | ◯ | ◯ | ◯ | ◯ | ◯ | ◯ |
| 40 | kHz | ◯ | ◯ | ◯ | ◯ | ◯ | ◯ | ◯ | ◯ | ◯ | ◯ |
| 50 | kHz | ◯ | ◯ | ◯ | ◯ | ◯ | ◯ | ◯ | ◯ | ◯ | ◯ |
| 60 | kHz | ◯ | ◯ | ◯ | ◯ | ◯ | ◯ | ◯ | ◯ | ◯ | ◯ |
| 70 | kHz | ◯ | ◯ | ◯ | ◯ | ◯ | ◯ | ◯ | ◯ | ◯ | ◯ |
| 79 | kHz | ◯ | ◯ | ◯ | ◯ | ◯ | ◯ | ◯ | ◯ | ◯ | ◯ |
| 80 | kHz | ◯ | ◯ | ◯ | ◯ | X | X | X | ◯ | ◯ | ◯ |
| 90 | kHz | ◯ | ◯ | ◯ | ◯ | X | X | X | ◯ | ◯ | ◯ |
| 100 | kHz | ◯ | ◯ | ◯ | ◯ | X | X | X | ◯ | ◯ | ◯ |
| 110 | kHz | ◯ | ◯ | ◯ | ◯ | X | X | X | ◯ | ◯ | ◯ |
| 120 | kHz | ◯ | ◯ | ◯ | ◯ | X | X | X | ◯ | ◯ | ◯ |
| 121 | kHz | ◯ | ◯ | ◯ | ◯ | ◯ | ◯ | ◯ | ◯ | ◯ | ◯ |
| 130 | kHz | ◯ | ◯ | ◯ | ◯ | ◯ | ◯ | ◯ | ◯ | ◯ | ◯ |
| 140 | kHz | ◯ | ◯ | ◯ | ◯ | ◯ | ◯ | ◯ | ◯ | ◯ | ◯ |
| 150 | kHz | ◯ | ◯ | ◯ | ◯ | ◯ | ◯ | ◯ | ◯ | ◯ | ◯ |

(◯: layer separation occurs, X: layer separation does not occur)

As can be seen from the results in Table 3, when the frequency of the ultrasonic wave applied according to the embodiment of the present invention in the first ultrasonication was set to 80 to 120 kHz, and the temperature of the mixture of the cannabidiol and ethanol was set to 40 to 60° C., the emulsified cannabidiol oil was not separated from the purified water at all.

### Experiment 4: Test for Occurrence of Layer Separation Upon Exposure to Ultraviolet Rays (UV)

The oil emulsions prepared in Example and Comparative Example were exposed to 150 nm-wavelength ultraviolet rays for 5 hours, a test was performed to determine whether or not layer separation between oil and purified water occurred, and the results are shown in Table 4 below (the frequency of ultrasonic waves applied in the first ultrasonication and the temperature conditions of the mixture are changed).

TABLE 4

| Item | | 10° C. | 20° C. | 30° C. | 39° C. | 40° C. | 50° C. | 60° C. | 61° C. | 70° C. | 80° C. |
|---|---|---|---|---|---|---|---|---|---|---|---|
| 30 | kHz | ◯ | ◯ | ◯ | ◯ | ◯ | ◯ | ◯ | ◯ | ◯ | ◯ |
| 40 | kHz | ◯ | ◯ | ◯ | ◯ | ◯ | ◯ | ◯ | ◯ | ◯ | ◯ |
| 50 | kHz | ◯ | ◯ | ◯ | ◯ | ◯ | ◯ | ◯ | ◯ | ◯ | ◯ |
| 60 | kHz | ◯ | ◯ | ◯ | ◯ | ◯ | ◯ | ◯ | ◯ | ◯ | ◯ |
| 70 | kHz | ◯ | ◯ | ◯ | ◯ | ◯ | ◯ | ◯ | ◯ | ◯ | ◯ |
| 79 | kHz | ◯ | ◯ | ◯ | ◯ | ◯ | ◯ | ◯ | ◯ | ◯ | ◯ |
| 80 | kHz | ◯ | ◯ | ◯ | ◯ | X | X | X | ◯ | ◯ | ◯ |
| 90 | kHz | ◯ | ◯ | ◯ | ◯ | X | X | X | ◯ | ◯ | ◯ |
| 100 | kHz | ◯ | ◯ | ◯ | ◯ | X | X | X | ◯ | ◯ | ◯ |
| 110 | kHz | ◯ | ◯ | ◯ | ◯ | X | X | X | ◯ | ◯ | ◯ |
| 120 | kHz | ◯ | ◯ | ◯ | ◯ | X | X | X | ◯ | ◯ | ◯ |
| 121 | kHz | ◯ | ◯ | ◯ | ◯ | ◯ | ◯ | ◯ | ◯ | ◯ | ◯ |
| 130 | kHz | ◯ | ◯ | ◯ | ◯ | ◯ | ◯ | ◯ | ◯ | ◯ | ◯ |
| 140 | kHz | ◯ | ◯ | ◯ | ◯ | ◯ | ◯ | ◯ | ◯ | ◯ | ◯ |
| 150 | kHz | ◯ | ◯ | ◯ | ◯ | ◯ | ◯ | ◯ | ◯ | ◯ | ◯ |

(◯: layer separation occurs, X: layer separation does not occur)

As can be seen from the results in Table 4, when the frequency of the ultrasonic wave applied according to the embodiment of the present invention in the first ultrasonication was set to 80 to 120 kHz, and the temperature of the mixture of the cannabidiol and ethanol was set to 40 to 60° C., the emulsified cannabidiol oil was not separated from the purified water at all.

Then, the conditions of the first ultrasonication were set to 100 kHz and 50° C. and the frequency of the ultrasonic wave applied during the second ultrasonication to perform a test to measure the emulsion conversion efficiency and determine whether or not the presence or absence of layer separation occurs.

### Experiment 5: Test for Measurement of Emulsification Efficiency

The conditions of the first ultrasonication were set to 100 kHz and 50° C. and the frequency of the ultrasonic wave applied during the second ultrasonication was changed. The ratio (%) at which the used cannabidiol oil is converted into an emulsion was measured using the oil emulsions thus prepared and the results are shown in Table 5 below.

TABLE 5

| Frequency | Emulsion conversion (%) |
|---|---|
| 110 kHz | 92 |
| 120 kHz | 93 |
| 130 kHz | 93 |
| 140 kHz | 93 |
| 149 kHz | 93 |
| 150 kHz | 100 |
| 160 kHz | 100 |
| 170 kHz | 100 |
| 180 kHz | 100 |
| 181 kHz | 94 |
| 190 kHz | 94 |
| 200 kHz | 94 |
| 210 kHz | 94 |
| 220 kHz | 94 |
| 230 kHz | 94 |
| 240 kHz | 94 |

As can be seen from the results of Table 5, the emulsion conversion efficiency of oil is 100% when the frequency of the ultrasonic waves applied in the second ultrasonication is set to 150 to 180 kHz according to the present invention.

As is apparent from the above description, the method for preparing a vegetable oil emulsion using a cannabidiol oil according to the present invention has an advantage of causing excellent oil-emulsion conversion efficiency and of preventing layer separation between cannabidiol and a polar solvent such as water even after a long period of time.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of preparing a vegetable oil emulsion using a cannabidiol oil comprising:
adding ethanol to a gel-type cannabidiol oil while heating the cannabidiol oil in a water bath (S100);
continuously performing the heating in the water bath to reduce a particle size of the cannabidiol oil in the result (S200);
stopping the heating in the water bath and adding purified water to the result (S300); and
heating the result again in a water bath while slowly elevating a temperature to completely remove the ethanol (S400).

2. The method according to claim 1, wherein step (S100) is performed by heating the result in the water bath at a temperature of 50 to 70° C.

3. The method according to claim 1, wherein step (S200) is performed until an average particle size of the cannabidiol oil reaches 20 nm or less.

4. The method according to claim 1, wherein a temperature of the purified water added in step (S300) is 15 to 20° C.

5. The method according to claim 1, further comprising:
applying ultrasonic waves to the cannabidiol oil mixed with purified water (S500) after step (S400).
